(19) United States
(12) Patent Application Publication
(10) Pub. No.: US 2026/0314955 A1
(43) Pub. Date: Oct. 15, 2026

(54) SAFETY DEVICE AND METHOD FOR DETECTING AN EMERGENCY SITUATION OF A PERSON

(71) Applicant: Adaptive Regelsysteme Gesellschaft m.b.H., Salzburg (AT)

(72) Inventors: Michael Altenbuchner, Salzburg (AT); Andreas Ortner, Salzburg (AT)

(21) Appl. No.: 19/122,433

(22) PCT Filed: Oct. 19, 2023
(86) PCT No.: PCT/EP2023/079145

§ 371 Date: Apr. 18, 2025

(30) Foreign Application Priority Data
Oct. 21, 2022 (AT) A50816/2022

(51) Int. Cl. A61B 5/11; A61B 5/00; G01P 15/18; G08B 21/04

(52) U.S. Cl. CPC A61B 5/1117; A61B 5/1122; A61B 5/6805; A61B 5/7282; A61B 5/746; G01P 15/18; G08B 21/0446

(57) ABSTRACT

The aim of the invention is to better prevent false activations in a safety device which detects an emergency situation of a person wearing the safety device via the movement state. This is achieved in that the acceleration of the person wearing the safety device is detected by means of an acceleration sensor on the safety device, and the detected acceleration is analyzed by an analysis unit in order to detect an emergency situation of the person from the acceleration time curve produced from the detected acceleration. In the event of a detected emergency situation, a safety action is initiated by the analysis unit, wherein the analysis unit initiates the safety action for the produced acceleration time curve after a period of time, said period of time being set on the basis of the detected acceleration.

The present invention relates to a safety device for detecting an emergency situation of a person wearing the safety device, wherein an acceleration sensor is provided on the safety device which detects an acceleration, preferably in 3-dimensional space, of the person wearing the safety device, wherein an analysis unit is provided on the safety device which analyzes the detected acceleration in order to detect an emergency situation of the person from the acceleration time curve produced therefrom and the analysis unit initiates at least one configured safety action in the event of a detected emergency situation. The invention also relates to an associated method for detecting an emergency situation of a person wearing the safety device.

If a person works in a safety-critical environment, safety devices are sometimes used which use movement sensors to detect critical or emergency situations. A safety-critical environment is, for example, an electrical installation, an electrical power transmission system, an industrial plant, etc. For example, in the event of a person's cardiac arrest, help should be called as quickly as possible. It may be a matter of minutes, even seconds, between the life and death of a person. If a person is seriously injured in another way, help must also be called, but it is not quite as time-critical as in the case of cardiac arrest. This is particularly important if the person is working alone. The safety device worn by the person often detects such emergency situations with the aid of movement sensors, such as position or acceleration sensors. A movement sensor detects a certain movement state, for example a person not moving, lying down, etc. Such a safety device initiates a pre-alarm—usually an acoustic one—when a certain movement state is detected. If there is no response to the pre-alarm, for example by a noticeable movement or pressing a button on the safety device, an alarm is initiated and help is called for, for example by acoustic and/or visual signals, by sending text messages, by sending emails or by voice calls. However, pre-alarms can occur very frequently, even when there is no critical situation. For example, if the person is sitting at a desk or in a meeting, a state of inactivity can be mistakenly identified as a critical movement state and a pre-alarm or even an alarm may be initiated. Sooner or later, this will become annoying. A common reaction of the person wearing the safety device is to switch off this safety function of the safety device or to switch off the entire safety device. If the person forgets to switch the safety function or the safety device back on, there is a potential danger because critical situations can no longer be detected. A known way to work around the problem is to combine immobility with a position sensor. Immobility is only reported when the person is lying down or is at a certain angle. However, there is no guarantee that a person will actually end up lying horizontally after a fall, nor that a particular emergency situation, such as a heart attack, will occur when the person is in a particular position, for example in a more or less sitting position.

There is therefore a need to improve a safety device and a method that detects an emergency situation of a person wearing the safety device via a movement state in this respect, in particular by better suppressing false activations.

For this purpose, the analysis unit is designed to initiate the safety action for the produced acceleration time curve after a period of time and to set the period of time on the basis of the detected acceleration.

Because the period of time is set on the basis of the acceleration, i.e. the current movement state, of the person, it is easier to react to the fact that a certain movement state exists, but there is still no emergency situation for the person. Previously, a fixed assignment between acceleration and period of time was provided. This meant that a compromise had to be made, which in certain situations led to false activations. Because of the procedure according to the invention the period of time is based on the acceleration and can therefore be better adapted to current movement states, which makes it easier to avoid false activations.

It is advantageous if the analysis unit sets the period of time the shorter the lower the currently detected acceleration is. This is based on the consideration that at low accelerations, for example when the person is (almost) motionless, it is more likely that the person is in an emergency situation than at higher accelerations. In the case of expected accelerations based on a person's normal movement, it can be concluded that there is no emergency situation at all. This can be mapped by the period of time thereby allowing the initiation behavior of the safety device to be specifically configured.

In an advantageous embodiment, it is provided that a first acceleration threshold value is provided in the analysis unit, to which a period of time is assigned, and the analysis unit initiates the safety action if the detected acceleration falls below the first acceleration threshold value during the period of time assigned to the acceleration threshold value. This is particularly easy to implement because only an acceleration threshold value with an assigned period of time needs to be specified. A comparison of the detected acceleration with an acceleration threshold value is also easy to implement.

In order to refine the initiation behavior, it can be provided that a first acceleration threshold value and a second acceleration threshold value are provided in the analysis unit, to each of which a period of time is assigned, and the analysis unit initiates the safety action if the detected acceleration falls below the first acceleration threshold value during the period of time assigned to the first acceleration threshold value or if the detected acceleration falls below the second acceleration threshold value during the period of time assigned to the second acceleration threshold value. Each acceleration threshold value can also be assigned its own safety action in order to initiate different safety actions.

In a particularly advantageous embodiment, a movement sensor is additionally provided on the safety device which detects a movement signal characterizing the movement state, which signal is different from the acceleration, and that the analysis unit additionally sets the period of time on the basis of the detected movement signal. This allows the period of time to be influenced even further. The underlying concept is that an acceleration in combination with a certain movement signal, which for example reflects a person's orientation, may not be critical, but in combination with another movement signal it may well be critical. This allows false activations to be suppressed even further.

This can be implemented by the analysis unit setting at least one acceleration threshold value on the basis of the movement signal.

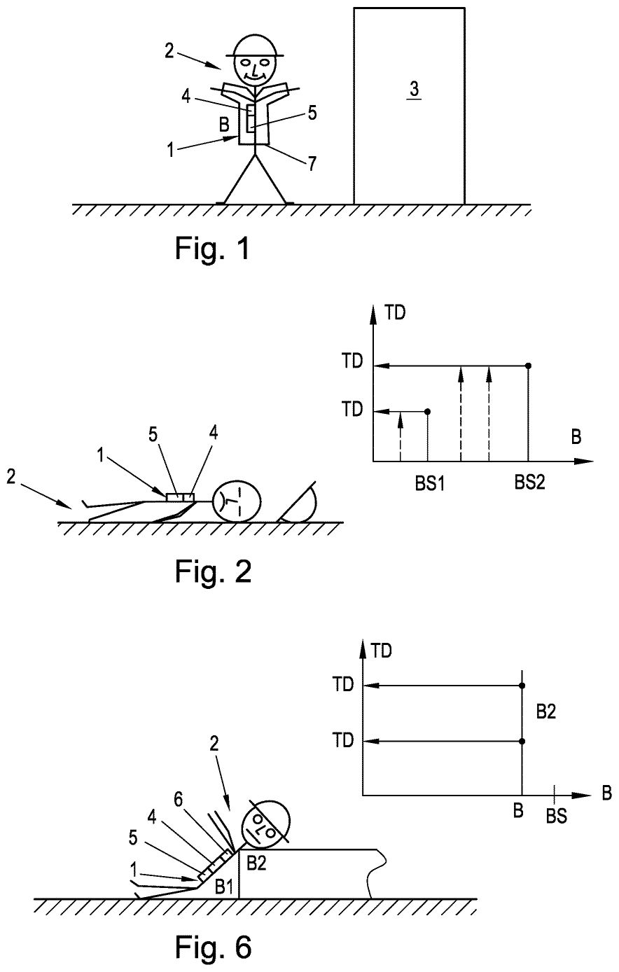

The present invention will be described in greater detail below with reference to FIG. 1 to 6, which by way of example show schematic and non-limiting advantageous embodiments of the invention. In the figures:

FIG. 1 shows a person with a safety device,

FIG. 2 shows a person with a safety device in an emergency situation,

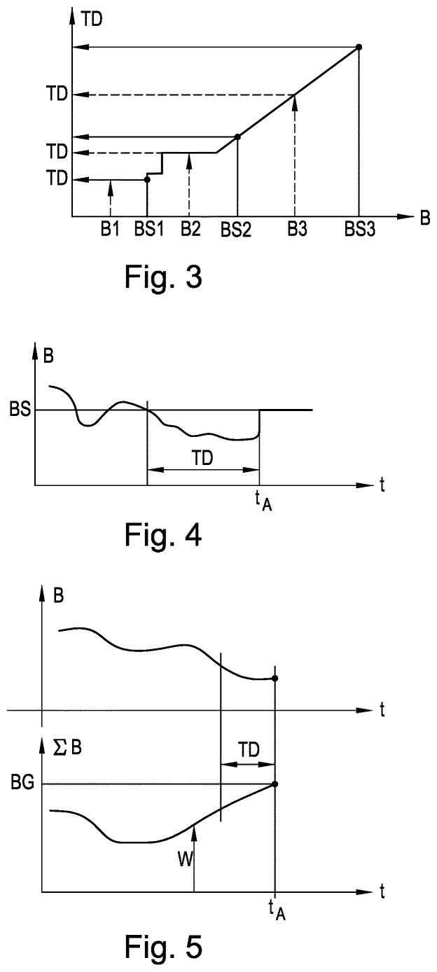

FIG. 3 shows an example of a relationship between acceleration and period of time,

FIG. 4 shows initiation when acceleration falls below an acceleration threshold value,

FIG. 5 shows initiation on the basis of a period of time resulting from an acceleration time curve, and

FIG. 6 shows a person with a safety device having an additional movement sensor.

The invention relates to a safety device 1 for detecting an emergency situation of a person 2 wearing the safety device 1, as shown in FIG. 1. An emergency situation is a situation in which there is a risk to the well-being of person 2, for example after an accident or a health emergency. The safety device 1 is placed on an item of clothing 7, such as a shirt, T-shirt, sweater, pants, etc., of the person 2, or is placed in a pocket of the item of clothing 7 or is otherwise worn by the person 2. The safety device 1 can also be integrated into the item of clothing 7, for example in the form of smart clothing with integrated electronics. For example, the person 2 is working on an installation 3, such as an energy distributor or an installation for generating, distributing or transmitting electrical energy. The installation 3 may be in a very remote location, for example a power pylon of an energy supply network.

An acceleration sensor 4 and an analysis unit 5 are provided on the safety device 1. The acceleration sensor 4 detects an acceleration of the person 2, preferably in 3-dimensional space, as the movement state of the person 2 wearing the safety device 1. The acceleration sensor 4 may be integrated into the safety device 1, but may also be present as a separate component and connected to the analysis unit 5 in a suitable manner.

The movement state is generally understood to mean the position (location), orientation, speed and/or acceleration of the person 2, usually in 3-dimensional space.

The analysis unit 5 may be microprocessor-based hardware on which corresponding software runs. However, the analysis unit 5 may also be designed as an integrated circuit, such as a field-programmable gate array (FPGA) or an application-specific integrated circuit (ASCI). Other implementations are also conceivable in principle.

The acceleration sensor 4, for example, determines accelerations in 3-dimensional space. The acceleration sensor 4, for example, provides three linear acceleration values for translational movement in the three spatial directions, but can also or additionally provide three rotational acceleration values for rotational movement. From this, a linear acceleration of the person 2 in space can be determined, but also, alternatively or additionally, a rotational acceleration state. The acceleration is usually related to a reference position or reference orientation, for example to a reference coordinate system. When reference is made below to an acceleration B of the person 2, a linear acceleration in space (or divided into the three spatial directions) and/or a rotational acceleration state of the person 2 is meant.

A so-called inertial measuring unit is often used to detect a movement state. As measured values, an inertial measuring unit provides three linear acceleration values for translational movement and three angular velocities for the rotation rates of a body (here the person 2). From these measured values, the speed of the person 2 in space (for example by integrating the linear accelerations), the position of the person 2 in space (for example by integrating the linear accelerations twice) and the orientation of the person 2 in space (by integrating the angular velocities) can be determined, i.e. a complete movement state of the person 2. The acceleration sensor 2 is advantageously designed as an inertial measuring unit.

The analysis unit 5 receives the acceleration B characterizing the movement state of the person 2 from the acceleration sensor 4 and analyses it in order to detect an emergency situation of the person 2 wearing the safety device 1 via the movement state. If an emergency situation is detected, the safety device 1, e.g. the analysis unit 5, initiates at least one configured safety action.

The safety action may comprise, for example, the setting off of an acoustic and/or visual alarm, but also the signaling of an alarm to a security center or security person configured in the safety device 1 or to an emergency service, for example by sending a text message, by sending an email or by making a voice call, etc. The safety device 1 therefore naturally also comprises a suitable communication interface in order to be able to set off the alarm. The communication interface is also based on the type of safety action and alarm.

The analysis unit 5 is configured to initiate the safety action after a period of time TD, wherein the analysis unit 5 sets the period of time TD on the basis of the detected acceleration. The period of time TD is thus adapted to the movement state of the person 2 in order to avoid unwanted false activations as far as possible. The concept according to the invention is that an emergency situation will only manifest itself with small accelerations, i.e. when the person 2 moves little or not at all. However, if higher accelerations B occur, for example during a person's normal movement, it is generally not to be assumed that the person 2 is in an emergency situation, or it can be assumed that the person 2 is still able to call for help.

At low accelerations B, the analysis unit 5 should initiate the safety action more quickly than at higher accelerations B. Therefore, the period of time TD in the analysis unit 5 is set shorter at low accelerations B than at higher accelerations B. If a certain acceleration B or an acceleration smaller than a certain acceleration B occurs during the period of time TD, the safety action is initiated. At higher or normal accelerations B, the period of time TD can become very long or even approach infinity.

This can of course be achieved in different ways, as explained below.

In an advantageous embodiment, the analysis unit 5 checks whether the acceleration B falls below a predetermined acceleration threshold value BS1 during the period of time TD.

The period of time TD can be assigned to the acceleration threshold value BS1, for example by configuration of the analysis unit 5. If the detected acceleration B falls below the acceleration threshold value BS1, a check is carried out to determine whether the acceleration B falls below during the period of time TD assigned to the acceleration threshold value BS1. Of course, multiple different acceleration threshold values BS1, BS2 can be provided, to each of which a period of time TD is assigned.

For example, the person 2 may be lying motionless on the ground as a result of an accident, as shown in FIG. 2. In such a movement state, only very small accelerations B are detected by the acceleration sensor 4, for example less than a first acceleration threshold value BS1 of 25 m-g (milli-g, i.e. thousandths of the acceleration due to gravity g). Even if the person 2 is motionless, it can be assumed that small accelerations B will be detected, either due to the measurement noise of the acceleration sensor 4 or due to vibrations of the ground on which the person 2 is lying. Therefore, a minimum acceleration threshold value BS1 should take such influences into account. Due to the lack of movement, this indicates a serious incident and a serious emergency situation for the person 2. In this case, the period of time TD of the undershoot can be set short, for example to 60 s (seconds). If this movement state does not change during the period of time TD, the configured safety action is initiated by the analysis unit 5, for example the setting off of an alarm, for example to an emergency service. However, if an acceleration B smaller than a second acceleration threshold value BS2 of 100 m-g is detected, it can be concluded that the person 2 is moving slightly, for example due to breathing. In this case, the second period of time TD2 of the undershoot can be set slightly longer, for example to 300 s (seconds). By the second acceleration threshold value BS2, for example, no safety action is initiated at all or only an alarm is initiated for a colleague, or further acceleration threshold values above the second acceleration threshold value BS2 can be specified.

FIG. 3 shows another exemplary embodiment of a relationship between acceleration and period of time TD. Shown are three different first movement signals B1, B2, B3, to each of which a period of time TD is assigned. In this example, there is a continuous (here linear) region and discrete regions of the assignment. For the smallest accelerations B, smaller than an acceleration threshold value BS1, a period of time TD is assigned to the acceleration threshold value BS1, which is to be taken into account for accelerations B1 below the acceleration threshold value BS1. For larger accelerations B3, the period of time TD is continuously based on the acceleration B3, i.e. each acceleration B3 is assigned a period of time TD according to a defined relationship. If the acceleration B3 is below the acceleration threshold value BS3, the period of time TD is set to the value resulting from the acceleration B3. For average accelerations B2, a mixture of a discrete and continuous assignment is provided, from which the period of time TD is derived.

The relationship between the acceleration B and the period of time TD, for example as shown in FIG. 2 or FIG. 3, is stored in a suitable manner in the analysis unit 5, for example in table form, as a mathematical function, discretely or continuously.

In this embodiment, a period of time TD is set based on a currently detected acceleration B and then it is checked whether the acceleration B has a certain time curve in this period of time, for example whether it is smaller than a certain acceleration threshold value BS. In other words, at the initiation time to of the safety action, the past acceleration curve in the past period of time TD has thus assumed a certain time curve, for example smaller than a certain acceleration threshold value BS. The past period of time TD is the period of time immediately before the initiation time t_{A}. This is shown as an example in FIG. 4, which shows the acceleration B over time t. At the initiation time t_{A} of the safety device 1, the acceleration B was less than an acceleration threshold value BS during the period of time TD, which is based on the acceleration B as described above.

In another embodiment, the period of time TD is not determined as such, but the period of time TD results from the acceleration time curve and is set in this way in the analysis unit 5. The period of time TD is therefore also based on the detected acceleration B.

In one possible embodiment, according to FIG. 5, the safety device 1 is initiated based on a period of time TD resulting from an acceleration B time curve.

For example, an acceleration effect W is determined from the acceleration B detected at each point in time (preferably in time-discrete sampling steps), for example as the reciprocal 1/B of the acceleration B, optionally also weighted, for example in order to take greater account of small accelerations. The acceleration effects W at each point in time are added up to an acceleration sum ΣB over a predetermined time window. The time window is a predetermined observation period, but can also be the entire period of time since the safety device 1 was switched on. At the same time, a predetermined acceleration effect W is subtracted from the current acceleration sum ΣB at each point in time, wherein the acceleration sum EB cannot become negative. For large accelerations B, the reciprocal is small and the acceleration sum EB will be low or even zero, in particular because an acceleration effect W is subtracted in each time step. If, however, a current acceleration B is small, the reciprocal is large and the acceleration sum ΣB increases sharply, in particular if the acceleration B remains small for a longer period of time, for example if the person 2 is motionless. If the acceleration sum EB exceeds a predetermined limit value BG, the safety action is initiated. This results in the period of time TD being the period before the initiation time ta during which the acceleration B had low values. The effect is therefore the same as in the embodiment shown in FIG. 4.

It is clear that the acceleration B in such a design can also be analyzed in a different way than with an acceleration sum ΣB. For example, a moving average could be determined over a time window. If the average is less than a limit value, the safety action could be initiated. Likewise, the area under the acceleration during a time window could be evaluated, for example as an integral.

In this embodiment, the period of time TD thus results from a past acceleration curve, wherein the period of time TD becomes shorter the lower the acceleration B in the past acceleration curve. The past acceleration curve is the acceleration B time curve in a predetermined time window before the current point in time.

The period of time TD may additionally be based on a second movement signal B2, which is different from the first movement signal B1, as explained with reference to FIG. 6.

In this exemplary embodiment, the safety device 1 comprises a movement sensor 6 which detects a movement state of the person 2 wearing the safety device 1. The movement sensor 6 detects a movement signal B2 which is different from the detected acceleration B. The second movement signal B2 is transmitted to the analysis unit 5 and analyzed there.

The movement sensor 6 is, for example, a position sensor that provides a movement signal B2 that characterizes the orientation of the person 2 in 3-dimensional space.

If the acceleration sensor 4 is designed as an inertial measuring unit that can also provide a position or orientation of the person 2 in space, the acceleration sensor 4 can of course also simultaneously function as a movement sensor 6. The acceleration sensor 4 and the movement sensor 6 are thus integrated into one sensor.

If the detected acceleration B falls below a specified acceleration threshold value BS, the additional movement signal B2 can also be checked. If the movement signal B2 is within a predetermined signal range, a certain period of time TD is set. If the movement signal B2 is outside the predetermined signal range, a different period of time TD is set, as indicated in FIG. 6. Of course, multiple signal ranges are also possible, each of which is assigned a certain period of time TD.

In the case of a position sensor as the second movement sensor 6, a range of −45° to +45° (relative to a horizontal) can be set as the signal range (position range), for example. If the acceleration B falls below an acceleration threshold value BS (in the above exemplary embodiment example, 100 m-g), the period of time TD is set to 60 s if, in addition, an orientation of the person 2 within the position range is determined. If the orientation is outside, the period of time TD is set to 300 s.

The period of time TD is therefore based not only on the detected acceleration B, but also on the further movement signal B2.

Furthermore, it is conceivable that the at least one acceleration threshold value BS is based on the second movement signal B2. In this case, it is possible to provide different acceleration threshold values BS for the acceleration B, on the basis of a current movement signal B2.

For example, a different acceleration threshold value BS could be provided when the person 2 is lying down, compared to a movement state in which the person 2 is standing or sitting. For example, the lying position of the person 2 can again be determined by a position range of −45° to +45°.

1. A safety device for detecting an emergency situation of a person wearing the safety device, comprising:
an acceleration sensor provided on the safety device which detects an acceleration of the person wearing the safety device;
an analysis unit provided on the safety device which analyzes the detected acceleration in order to detect an emergency situation of the person from an acceleration time curve produced therefrom and the analysis unit initiates at least one configured safety action in the event of a detected emergency situation, wherein the analysis unit is configured to initiate the safety action for the produced acceleration time curve after a period of time and to set said period of time on the basis of the detected acceleration.

2. The safety device according to claim 1, wherein the analysis unit sets the period of time to be shorter the lower the currently detected acceleration is.

3. The safety device according to claim 1, wherein a first acceleration threshold value is provided in the analysis unit, to which a period of time is assigned, and the analysis unit initiates the safety action if the detected acceleration falls below the first acceleration threshold value during the period of time assigned to the acceleration threshold value.

4. The safety device according to claim 1, wherein first acceleration threshold value and a second acceleration threshold value are provided in the analysis unit, to each of which a period of time is assigned, and the analysis unit initiates the safety action if the detected acceleration falls below the first acceleration threshold value during the period of time assigned to the first acceleration threshold value or if the detected acceleration falls below the second acceleration threshold value during the period of time assigned to the second acceleration threshold value.

5. The safety device according to claim 1, wherein a first acceleration threshold value and a second acceleration threshold value are provided in the analysis unit, to each of which a period of time is assigned, and the analysis unit initiates a first safety action if the detected acceleration falls below the first acceleration threshold value during the period of time assigned to the first acceleration threshold value, or the analysis unit initiates a second safety action if the detected acceleration falls below the second acceleration threshold value during the period of time assigned to the second acceleration threshold value.

6. The safety device according to claim 1, wherein the analysis unit is configured to initiate a safety action on the basis of a period of time resulting from an acceleration time curve.

7. The safety device according to claim wherein a movement sensor is provided on the safety device, which detects a movement signal characterizing the movement state, which signal is different from the acceleration, and in that the analysis unit sets the period of time additionally on the basis of the detected movement signal.

8. The safety device according to claim 7, wherein the analysis unit sets at least one acceleration threshold value on the basis of the movement signal.

9. The safety device according to claim 7, wherein the movement sensor is a position sensor and the movement signal indicates an orientation of the person.

10. A method for detecting an emergency situation of a person with a safety device worn by the person, comprising:
detecting an acceleration of the person wearing the safety device is by means of an acceleration sensor on the safety device;
analyzing the detected acceleration by an analysis unit in order to detect an emergency situation of the person from the acceleration time curve produced from the detected acceleration; and
initiating at least one configured safety action by the analysis unit in the event of a detected emergency situation, wherein the analysis unit initiates the safety action for the produced acceleration time curve after a period of time, wherein the period of time is set on the basis of the detected acceleration.

11. The method according to claim 10, wherein the period of time is set to be shorter the lower the currently detected acceleration is.

12. The method according to claim 10, wherein the safety action is initiated when the detected acceleration falls below the first acceleration threshold value during a period of time assigned to a predetermined acceleration threshold value.

13. The method according to claim 10, wherein the safety action is initiated if the detected acceleration falls below the first acceleration threshold value during a first period of time assigned to a predetermined first acceleration threshold value or if the detected acceleration falls below the second acceleration threshold value during a second period of time assigned to a predetermined second acceleration threshold value.

14. The method according to claim 10, wherein first safety action is initiated when the detected acceleration falls below the first acceleration threshold value during a first period of time assigned to a predetermined first acceleration threshold value, or a second safety action is initiated when the detected acceleration falls below the second acceleration threshold value during a second period of time assigned to a predetermined second acceleration threshold value.

15. The method according to claim 10, wherein safety action is initiated on the basis of a period of time resulting from an acceleration time curve.

16. The method according to claim 10, wherein a movement sensor detects a movement signal characterizing the movement state of the person, which signal is different from the acceleration, and in that the period of time is set additionally on the basis of the detected movement signal.

17. The method according to claim 16, wherein at least one acceleration threshold value is set on the basis of the movement signal.
